(19) United States
(12) Patent Application Publication
(10) Pub. No.: US 2010/0198125 A1
(43) Pub. Date: Aug. 5, 2010

(54) Manageable Arm and Hand Elevation Device

(76) Inventor: Michael Edward Todd, Rocklin, CA (US)

Correspondence Address: Michael E. Todd, 6423 Cormorant Circle, Rocklin, CA, 95765, US

(21) Appl. No.: 12/363,716

(22) Filed: Jan. 30, 2009

(51) Int. Cl. A61F 5/00

(52) U.S. Cl. 602/20

(57) ABSTRACT

A device for supporting and elevating the arm and hand above the heart while healing. The device comprises mainly of a belt, support pole, and straps for the arm and forearm. The support pole can be attached to different areas of the belt to support different waist line sizes along with left or right arm and/or hand injuries. The support pole is also adjustable for different human height, arm width and lengths. The device enables shoulder mobility and is supported by the waist, thus allowing the person to move around freely.

### BACKGROUND OF INVENTION

Appendage elevation is an important part of recovery and pain management for many injuries. The only devices available today that elevate the arm and hand over a person's heart require the use of an external object that is either immovable or cumbersome to move. There is a need for a device that provides shoulder mobility and allows the person to easily move from one area to the next while attempting to live a normal life.

Common devices that have been used for this application are slings that require an external object to hook to and raise the arm and hand above the heart. Other alternatives are pillows or foam that require a fixed object like a table or chair to lie on top of. Typically in hospitals, a fixed apparatus makes sense, but the lack of mobility is confining and can cause discomfort. Moderate shoulder and arm mobility prevents bruising on the arm and muscle strain in the shoulder that can be caused by a fixed apparatus.

Some of the aforementioned devices make it difficult to care for and even sometimes visually inspect the injured area. The present invention enables the patient to see and care for the injured arm and/or hand. The arm and forearm straps only cover a small portion of the body and they are easy to detach. This allows the patient to sterilize the injured area while elevated. This is very important in the cases that require an external fixation device that must be screwed into the bone. Daily sanitation must occur on the areas that penetrate the skin.

To elevate the arm and hand over the heart using a sling, the patient must stay in the place where the sling is attached. If the attached object is movable, the patient would have to carry the object With their healthy hand and arm. This is assuming the individual has one healthy arm and hand free to use. The pillow or foam pad solution requires a fixed object that is positioned in a way that is comfortable to the arm. If the patient decides to move about, they would have to support their arm and/or hand above their heart. This will cause shoulder strain over a long enough period of time. A need exist for a device that provides mobility and freedom to move about while keeping the arm and hand elevated above the heart with little to no effort by the user.

### BRIEF DESCRIPTION OF DRAWINGS

The present invention is illustrated by way of example, and not by limitation, in the figures of the accompanying drawings in which elements having the same reference numeral designations represent like elements throughout and wherein:

FIG. 1 is a representation of a person using the device with allowed mobility

FIG. 2 is an exemplary of the adjustable belt along with support and fasteners

FIG. 3 is an exemplary of the adjustable pole along with adjustable straps

### DETAILED DESCRIPTION

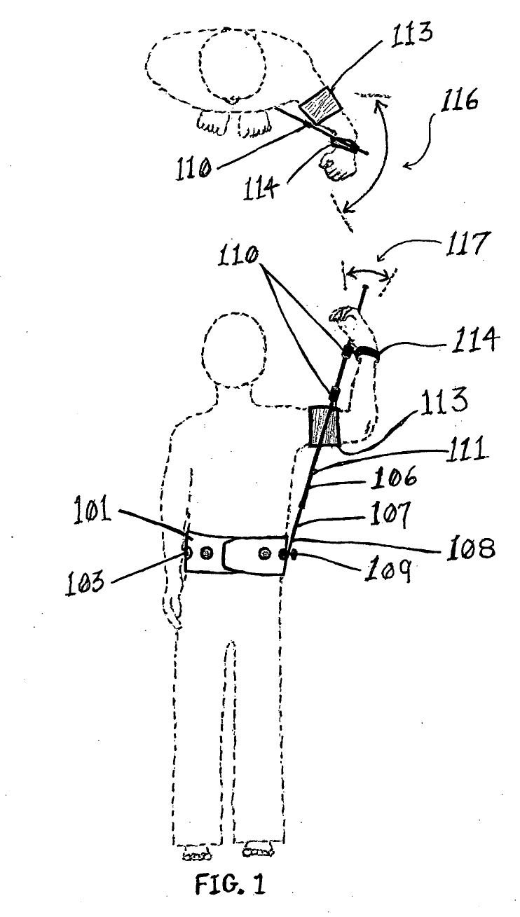

As shown in FIG. 1, the present invention allows moderate shoulder and arm mobility 116 and 117 while keeping the arm and hand above the heart. The present invention is self contained by strapping the belt 101 around a person's waist. The belt is made from multiple layers of neoprene and fabric 101. In between the layers are six plastic inserts 102 that provide support. They are separate pieces to provide flexibility in between the pieces, but are necessary for support and added comfort.

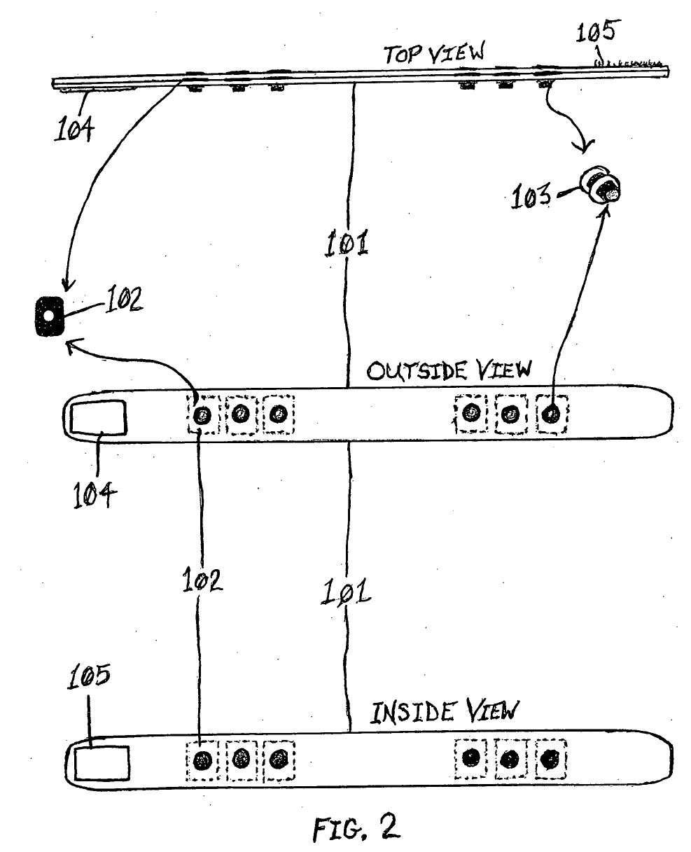

FIG. 2 represents the belt 101 structure and components. 103 are two metal discs that mount to the inside and outside layers of the belt 101 and pierce through the plastic inserts 102 that provide support. The mounting discs 103 are threaded in the center enabling the adjustable pole to be fastened to the belt 101 using the mounting screw 109. This is why there is a hole in the center of 102. The belt 101 and its components are the foundation of the manageable arm and hand elevation device. This provides the user his or her ability to move around freely without dependence of an external object.

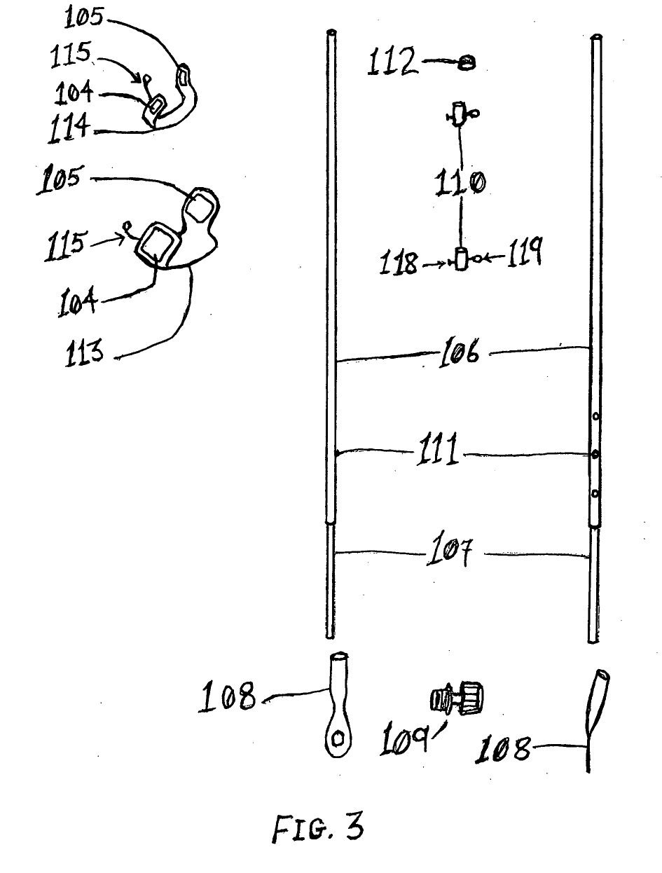

FIG. 3 shows the mounting screw 109 that is inserted into the eyehole of the metal slider 108 attached to the end of the inner pole 107. The metal slider 108 is the component that allows for the arm and shoulder mobility while keeping the arm and hand above the heart. 108 is permanently attached to the inner pole 107. The inner pole 107 contains a spring loaded button 111 that fixes the adjustment of the inner pole 107 to the holes on the outer pole 106. This allows the pole to be adjusted to the height of the user and can be modified anytime by pressing the button 111. There is a rubber cap 112 that fits to the top of the outer pole 106. This is used for protection purposes only by softening the end of the pole.

As demonstrated in FIG. 3, additional attachments to the outer pole 106 are two sleeves 110 that can be fixed to the pole by a fastening screw 118. The bottom sleeve 110 is used to attach the arm strap 113 via the loop screw 119. The top sleeve 110 is for the forearm strap 114 and attaches to the loop screw 119 via the straps attachment 115. These sleeves 110 are allowed to move up and down the outer pole 106 to fit to different arm lengths. When the device is fitted to the user, these components should be fastened in place. Each arm strap contains a Velcro hook side 105 and Velcro loop side 104 that enables the user to attach the straps to different width arms and forearms.

The belt 101 is adjustable by attaching the Velcro hook side 105 to the Velcro loop side 104 in different places depending on the person's waistline size. The mounting discs 103 are available in six places on the left and right side of the belt. This supports different waistline sizes as well as left or right arm and/or hand injuries. The manageable arm and hand elevation device can be quickly attached and detached by pushing or pulling the Velcro on 101, 114, and 114.

I claim:

1. A device that elevates the human arm and hand above his or her heart without any dependency of external object while allowing for shoulder movement and adjustable to fit different sized people comprising of:
an adjustable belt with built-in pole support and locking features;
said adjustable pole with adjustable arm and forearm support straps;
said arm and forearm support straps adjust to a person's arm length and width;
a locking screw that enables the said pole to be mounted to different areas of the said belt serving the left or right arm and preference of arm position;
the said pole is able to swivel on the locking screw allowing shoulder mobility.

2. A device that elevates the human arm and hand comprising of:
a belt that fits to his or her waistline with a pole attached to said belt;
said pole attaches to his or her arm.

3. A device that elevates the human arm and hand above his or her heart comprising of:
an adjustable belt that fits his or her waistline;
an adjustable pole that attaches to said belt;
adjustable arm straps that attach to said pole.
